(19) United States
(12) Patent Application Publication
(10) Pub. No.: US 2025/0366823 A1
(43) Pub. Date: Dec. 4, 2025

(54) METHODS AND SYSTEMS FOR ULTRASOUND PROBE CLEANING

(71) Applicant: GE Precision Healthcare LLC, Waukesha, WI (US)

(72) Inventor: Christian Perrey, Linz (AT)

(21) Appl. No.: 18/678,825

(22) Filed: May 30, 2024

(51) Int. Cl. A61B 8/00

(52) U.S. Cl. CPC A61B 8/4422; A61B 8/4254; A61B 8/461; A61B 8/467

(57) ABSTRACT

Systems and methods are provided for an ultrasound system. An ultrasound system includes an ultrasound probe, a holder in which the ultrasound probe is positioned when the ultrasound system is not in an active use state, and a processor communicatively coupled to a display and a user interface. The processor includes instructions stored on non-volatile memory, that when executed cause the processor to determine an operating state of the ultrasound system and in response to determining the operating state is not the active use state, automatically determine by image analysis if a probe face of the ultrasound probe is clean, and in response to determining the probe face is not clean, perform a processor operation to indicate the probe face is not clean.

### TECHNICAL FIELD

Embodiments of the subject matter disclosed herein relate to systems and methods for automatically determining presence of residual gel on an ultrasound probe before and after an ultrasound exam.

### BACKGROUND

During an ultrasound exam, a gel may be applied between an ultrasound probe and the object being examined to facilitate transmission of acoustic waves between the ultrasound probe and the object. Conventionally, it is up to the user to remember to wipe the ultrasound probe clean of gel after the exam. Residual gel left behind on the ultrasound probe between examinations may result in growth of bacteria and potential cross patient contamination.

### BRIEF DESCRIPTION

In one embodiment, an ultrasound system comprises an ultrasound probe comprised of transducer elements comprised of piezoelectric material, a damping block positioned behind the transducer elements, a matching layer positioned in front of the transducer elements, and a sensor configured to detect motion of the ultrasound probe; a holder comprising a sensor configured to detect motion of the ultrasound probe into and/or out of the holder, wherein the ultrasound probe is positioned inside the holder when the ultrasound system is not in an active use state; and a processor communicatively coupled to a display and a user interface, wherein the processor includes instructions stored on non-volatile memory that when executed cause the processor to: determine an operating state of the ultrasound system; in response to determining the operating state is not the active use state, automatically determine by image analysis if a probe face of the ultrasound probe is clean; and in response to determining the probe face is not clean, perform a processor operation to indicate the probe face is not clean.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, herein below:

FIG. 1 shows a block diagram of an ultrasound system.

FIG. 2 shows an illustration of an ultrasound probe.

FIG. 3 shows a flowchart of an overview of a method for warning an ultrasound user of a dirty ultrasound probe.

FIG. 4 shows a flowchart of method for automatically determining a mode of an ultrasound system.

FIG. 5 shows a flowchart of a detailed method for warning an ultrasound user of a dirty ultrasound probe.

FIG. 6 shows an example of a near field image acquired with a clean dry ultrasound probe.

FIG. 7 shows an example of a near field image acquired with a wet ultrasound probe.

FIG. 8 shows an example of a near field image acquired with a dirty ultrasound probe including a small amount of residual gel.

FIG. 9 shows an example of a near field image acquired with a dirty ultrasound probe including a large amount of residual gel.

FIG. 10 shows an example of a displayed warning on a monitor of an ultrasound system.

FIG. 11 shows an example of a flowchart of a method for automatic image analysis to determine if an ultrasound probe is clean.

FIG. 12 shows a block diagram of a machine learning algorithm for determining if an ultrasound probe is clean.

### DETAILED DESCRIPTION

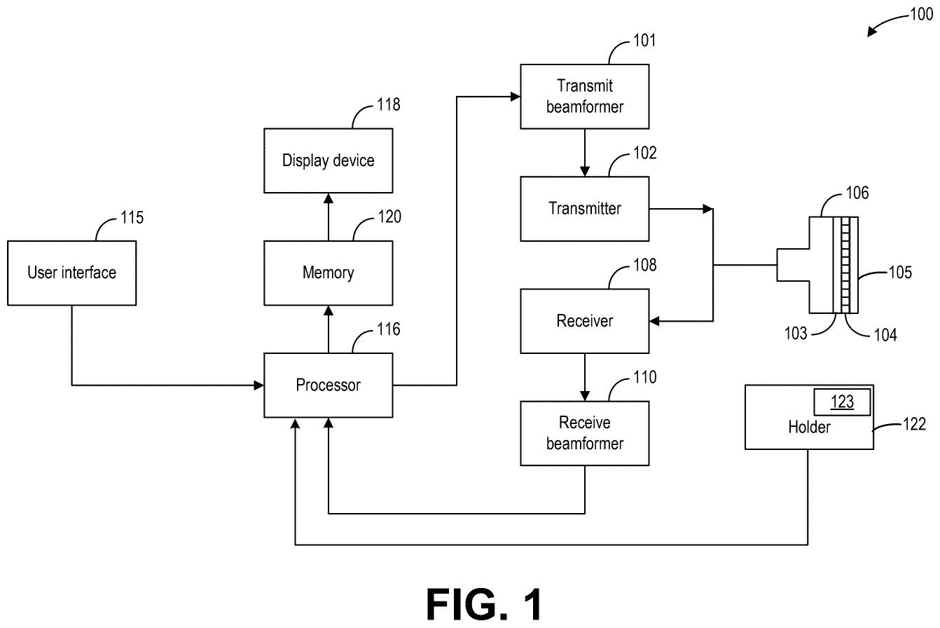

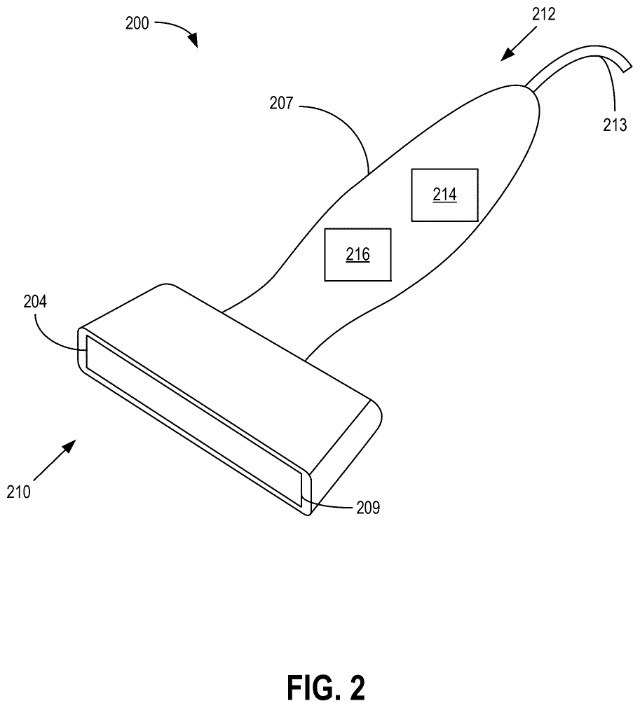

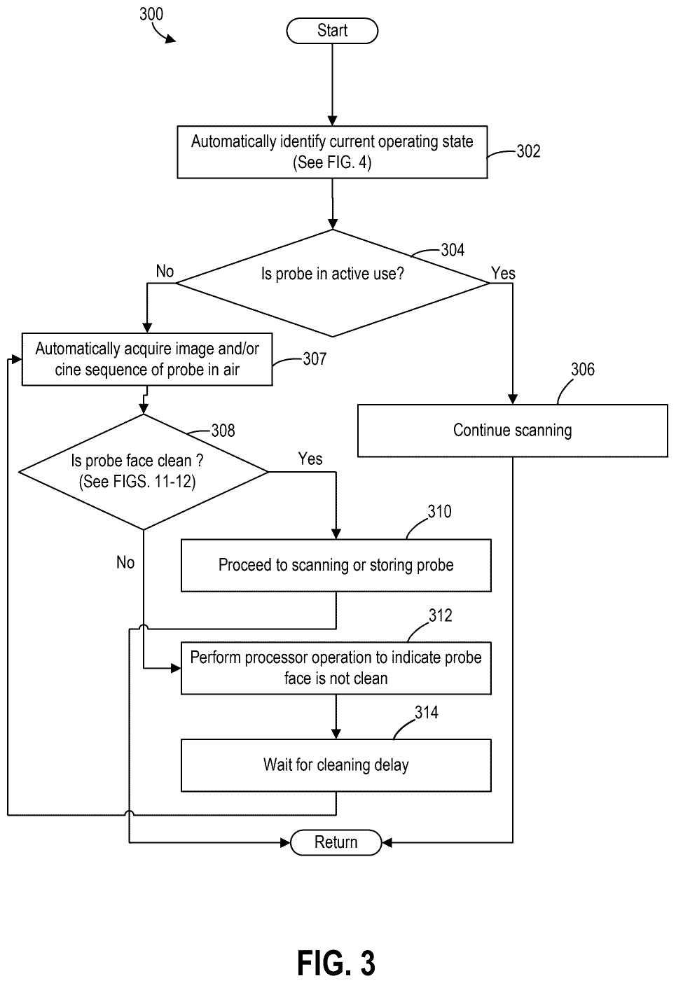

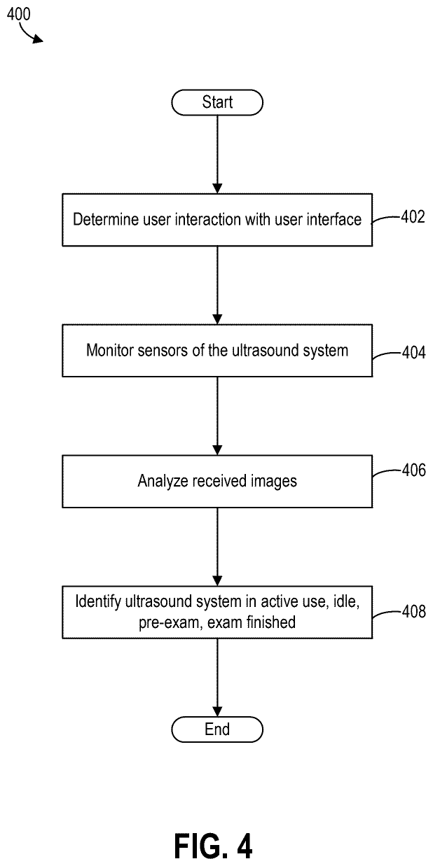

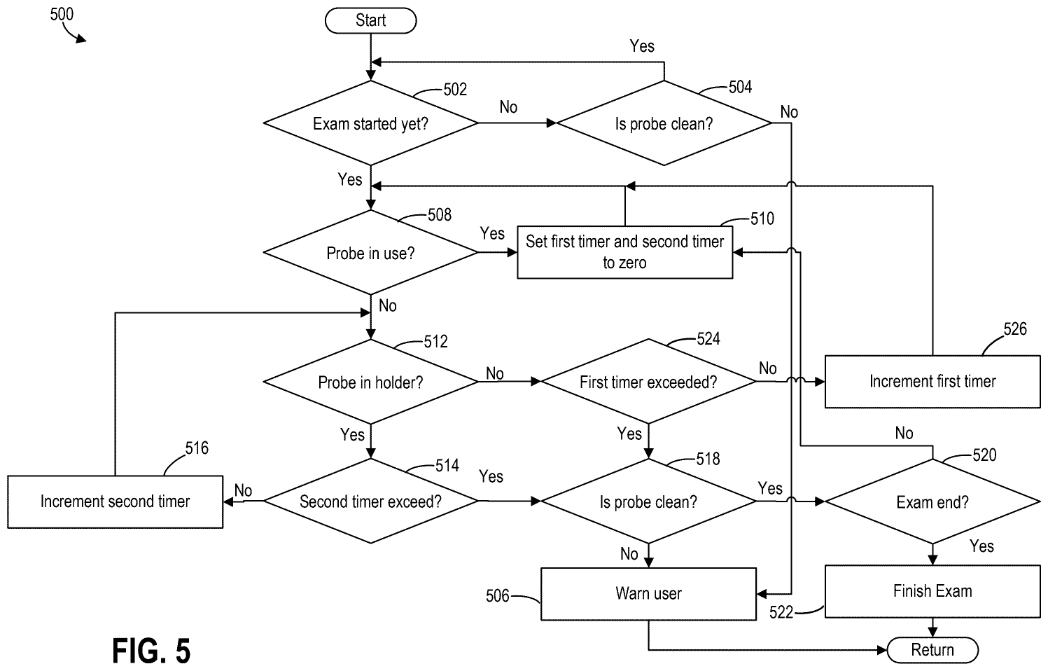

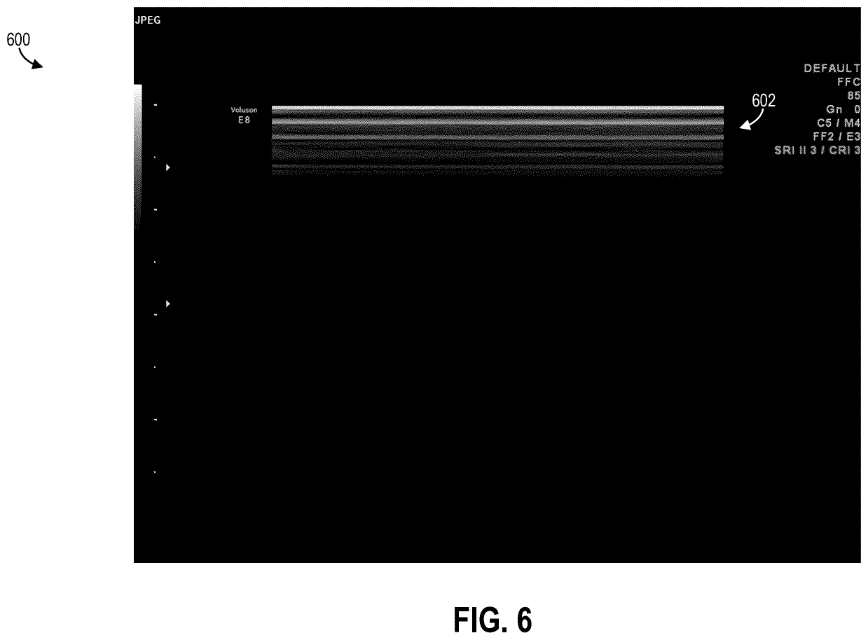

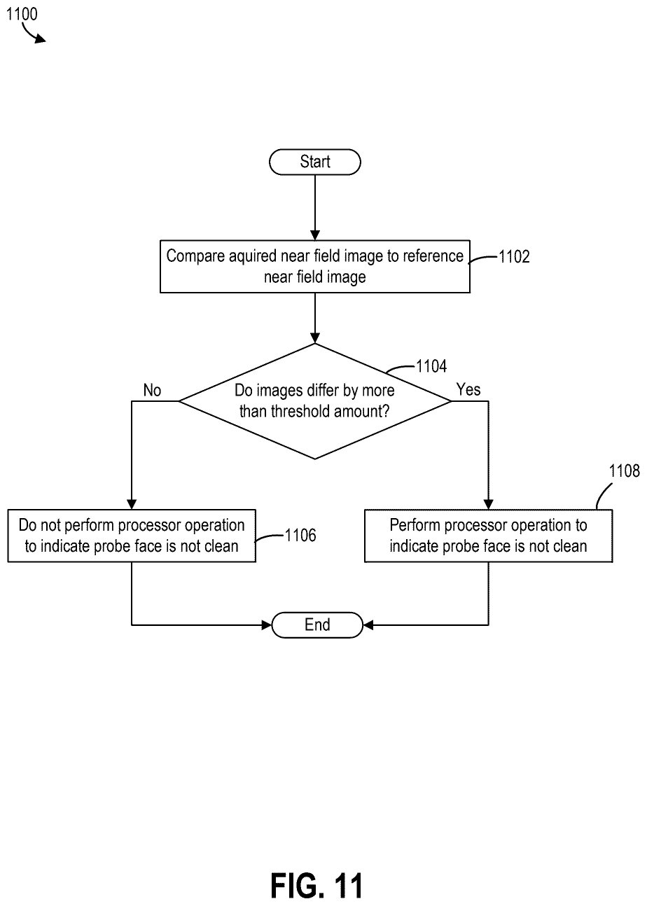

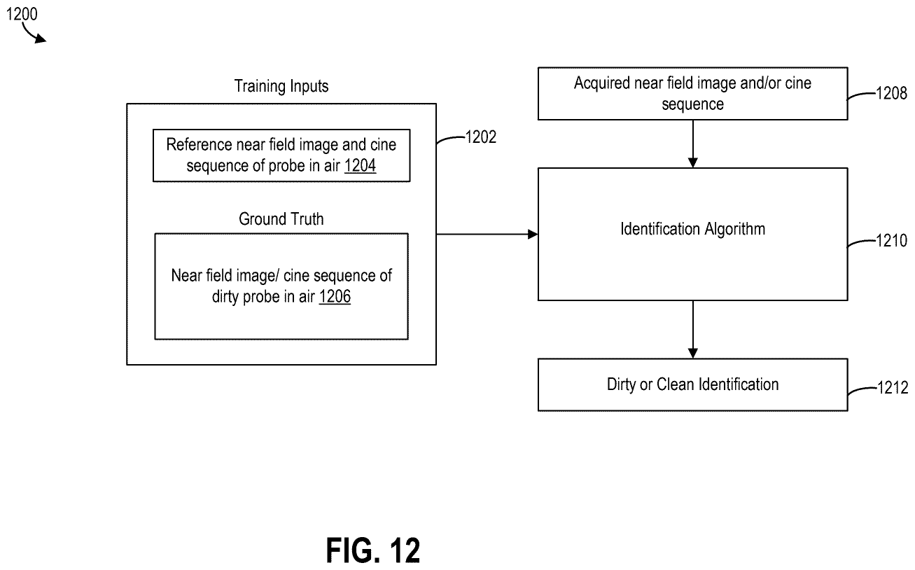

The following description relates to systems and methods for automatically maintaining a clean, gel and water free surface of an ultrasound probe between ultrasound exams. An ultrasound exam may be performed with an ultrasound system including an ultrasound probe, such as the ultrasound imaging system shown schematically in FIG. 1. The ultrasound imaging system may include a handheld ultrasound probe as shown in FIG. 2. A gel may be applied on a patient in the area being imaged to aid in transmission of acoustic waves to and from the ultrasound probe. Once dispensed, the gel is introduced to a non-sterile environment and may become host to living contaminants. If the gel is not removed from a surface of the ultrasound probe between patients, the contaminants may be transferred to the patient, and the patient may experience poor health outcomes as a result. Conventionally, it is up to the user (e.g., ultrasound technician) to remember to remove gel from the ultrasound probe after an exam to ensure that the ultrasound probe is free of old gel before starting a new exam. A system that may automatically warn the user before or after an exam that there is residual material (e.g., gel or water) on a contact surface of the ultrasound probe can significantly reduce the chances of using a contaminated ultrasound probe for an exam due to human error. Additionally, detecting presence of gel and/or water residual on a probe surface by image analysis as describe herein may detect residuals that may otherwise be missed by the human eye. A high level method for automatically warning the user to clean the ultrasound probe is shown in FIG. 3. Part of the method is to first automatically determine a state of use of the ultrasound system. Methods for automatically determining if an ultrasound system is about to be used, currently in use, or just finishing an exam are shown in FIG. 4. Further, the system may automatically determine if residual gel is present based on near-field image or cine sequence. Examples of near field images acquired by ultrasound probes including different levels and types of contamination are shown in FIGS. 6-9. Methods for automatically determining the presence of residual gel are shown in FIGS. 11-12. A method, as detailed further in FIG. 5, may combine automatically detecting a state of the ultrasound system with automatically detecting a contaminant on the ultrasound probe to stop an ultrasound exam and/or to warn the user to clean the probe before continuing. In this way, the warning to clean the ultrasound probe may be automatically generated when demanded and may not be generated when not demanded. For example, the warning may not be demanded if the ultrasound probe is clean or if the exam is in progress. In this way, user compliance may be increased as compared to merely displaying a routine reminder at regular intervals whether cleaning is demanded or not, which the user may become habituated to ignore.

Referring to FIG. 1, a schematic diagram of an ultrasound imaging system 100 in accordance with an embodiment of the disclosure is shown. Herein, ultrasound imaging system 100 may also be referred to as ultrasound system 100. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. As explained further below, the transducer elements 104 may be comprised of a piezoelectric material. When a voltage is applied to a piezoelectric crystal, the crystal physically expands and contracts, emitting an ultrasonic spherical wave. In this way, transducer elements 104 may convert electronic transmit signals into acoustic transmit beams. The probe 106 may further include a damping block 103 positioned behind the transducer elements 104 (e.g., closer to a handle of the probe) and adapted to absorb ultrasound waves propagating backward from the transducer elements 104. Additionally, the probe 106 may include a matching layer 105 positioned in front of (e.g., closer to the probe face) the transducer elements 104 and adapted to provide an acoustic impedance gradient for the ultrasound waves to smoothly penetrate into the subject and for reflected ultrasound waves to smoothly return to the transducer elements for detection.

After the elements 104 of the probe 106 emit pulsed ultrasonic signals into a body (of a patient), the pulsed ultrasonic signals are back-scattered from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data. Additionally, transducer element 104 may produce one or more ultrasonic pulses to form one or more transmit beams in accordance with the received echoes.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. In one embodiment, data acquired via ultrasound system 100 may be used to train a machine learning model. A user interface may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by augmenting the data as described further herein, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

The ultrasound system 100 may also include a holder 122. Probe 106 may be positioned inside holder 122 and may rest within holder 122 when not in active use. In some examples, ultrasound system 100 may include more than one probe 106 and in those examples a holder 122 may be included for each ultrasound probe 106. Holder 122 may include one or more sensors 123 communicatively coupled to processor 116. The sensors may be configured to detect motion of probe 106 both into and out of holder 122. In this way, processor 116 may determine if probe 106 is in use.

In various embodiments of the present disclosure, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (i.e., pixels) in the resulting image. These missing pixels occur because each element of the two-dimensional block should typically cover many pixels in the resulting image. For example, in current ultrasound imaging systems, a bicubic interpolation is applied which leverages neighboring elements of the two-dimensional block. As a result, if the two-dimensional block is relatively small in comparison to the size of the bitmap image, the scan-converted image will include areas of poor or low resolution, especially for areas of greater depth.

FIG. 2 is a schematic perspective view of an ultrasound probe 200 in accordance with an embodiment. The ultrasound probe 200 may be similar to, or the same as, the ultrasound probe 106 described above with reference to FIG. 1. The ultrasound probe 200 shown in FIG. 2 is a linear probe. Elements 204 (which may be similar to, or the same as, elements 104 described above with reference to FIG. 1) are arranged in a linear array. The ultrasound probe 200 may have a different configuration according to various embodiments. For example, the ultrasound probe 200 may be a curved array probe or a linear array probe. An array face 209 and elements 204 are positioned at a first end 210 of the ultrasound probe 200 and opposite to a handle 207 arranged at a second end 212 of the ultrasound probe 200. The array face 209 may contact a skin of the patient during an ultrasound exam and may retain some gel after the patient exam. Herein, array face 209 may also be referred to as a probe face or a probe surface. The ultrasound probe 200 may further include sensors 214. Sensors 214 may include a motion sensor configured to sense movement of the ultrasound probe and communicatively coupled to a controller of the ultrasound system (e.g., processor 116 of FIG. 1). Additionally, the ultrasound probe 200 may further include a vibration motor 216. Vibration motor 216 may be configured to vibrate handle 207 to draw the attention of the user, such as when the ultrasound system determines the array face 209 may not be clean, as discussed further below. Further, ultrasound probe 200 may rest in a holder such as holder 122 when not in use. Sensors 214 and/or sensor included in the holder may be configured to determine if ultrasound probe 200 is resting in the holder or positioned elsewhere. In some examples, the ultrasound probe 200 may be configured to communicate wirelessly with an electronic controller of an ultrasound imaging system. In other examples the ultrasound probe 200 may be configured with a cable, such as cable 213, and may communicate electronically with the controller via the cable 213.

An ultrasound system, such as ultrasound system 100 including an ultrasound probe such as probe 106 or probe 200 may demand a presence of gel between the ultrasound probe and subject being examined. A user of the ultrasound system (e.g., ultrasound technician) may be have a static reminder or be reminded at regular intervals to clean gel from the surface of the ultrasound probe when an exam is finished and to check for presence of residual gel before starting an imaging procedure. However, static or regular intervals may easily become background noise, especially to a skilled user. Instead, an automatic triggered warning method may combine automatically detecting a state of the ultrasound system and a cleanliness of the ultrasound probe to remind the user to clean the ultrasound probe when cleaning is demanded and may not display a warning when cleaning is not demanded. In this way, compliance with the cleaning may be increased and adverse health events due to patient exposure to contaminated ultrasound probes may be decreased.

Turning now to FIG. 3, a flowchart of a method 300 is shown. Method 300 provides an overview of an automatic triggered probe cleaning warning method. Method 300 and other methods described herein may be carried out by a processor, such as the processor 116 of the ultrasound imaging system 100 of FIG. 1, in accordance with instructions stored in non-volatile memory of a computing device, such as memory 120 of ultrasound imaging system 100 of FIG. 1.

At 302, method 300 includes automatically identifying a current operating mode of the ultrasound system. In some examples, the operating mode of the ultrasound system may include, but is not limited to, an exam start before active probe use, active probe use, idle probe, an exam finished. Automatically identifying current operating mode may proceed automatically in response to the ultrasound system being powered on. In some examples, automatically identifying a current operating mode may be based on signals received from sensors of the ultrasound probe, such as sensors 214 of FIG. 2. An example of a method for automatically identifying a current operating state of the ultrasound system is described further below with respect to FIG. 4.

Turning briefly to FIG. 4, a flowchart of a method 400 of determining an operating state of the ultrasound system is shown. The steps of method 400 may occur automatically upon powering on the ultrasound system. Method 400 may be executed at step 302 of method 300 described above with respect to FIG. 3.

At 402, method 400 includes determining the user interaction with the user interface, such as user interface 115 of FIG. 1. Determining the user interaction with the user interface may include determining features or options of the user interface. For example, a user accessing a field of the user interface to enter patient information may indicate that the ultrasound system is in an pre-exam state and not in the active use state. As a further example, the user may interact with the user interface by selecting options for scanning which indicates that ultrasound system is in an active scan state. As an additional example, the user may interact with the user interface to request a report, requesting the report may indicate that the ultrasound system is in an exam finished state.

Method 400 proceeds to 404 and includes monitoring sensors of the ultrasound system. As one example, a sensor of the ultrasound probe may be a motion sensor of the ultrasound probe, such as sensor 214 of FIG. 1. Monitoring the motion sensor may determine if the ultrasound probe is currently being moved. An actively moving ultrasound probe may indicate that the operating state of the ultrasound system is active use. As an additional example, the sensors may include a holder sensor as described above with respect to holder 122. The holder sensor may be configured to detect movement of the ultrasound probe into and out of the holder. If the ultrasound probe is moved into the holder, the ultrasound system state may be idle or finished. If the ultrasound probe is moved out of the holder, the ultrasound system may be idle or in the active state.

At 406, method 400 includes analyzing received images from the ultrasound probe. As one example, a cine sequence of images received may be analyzed to determine if the image is moving over time. Movement in the cine sequence may indicate that the ultrasound exam is in progress and the ultrasound system is in an active use state. As an alternate example, an intensity of far field images may be analyzed. Intensity below a threshold intensity in the far field image may indicate that an ultrasound probe is not acoustically coupled to a subject, therefore indicating the ultrasound system maybe in an idle or exam finished state. Intensity above the threshold intensity in the far field image may indicate the ultrasound system is in an active use state.

At 408, method 400 includes identifying a state of the ultrasound system as being an active use state, idle state, pre-exam state, or exam finished state. Identifying the state of the ultrasound system may be based on the inputs acquired at steps 402, 404, and 406 of method 400. In some examples, a state that is not the active use state (e.g., the idle state, pre-exam state, and exam finished state) may be referred to as an inactive state. For example, a combination of the user interacting with scan settings of the user interface and motion indicated by the motion sensor of the ultrasound probe may indicate the ultrasound system is in an active use state. As another example, a combination of the user interacting with scan settings but the image analysis determining an intensity below the threshold intensity may indicate the ultrasound system is in an idle state. The identified state may then be used to determine whether or not to check if the probe is clean as described in FIG. 3.

Returning now to method 300 of FIG. 3, at 304, method 300 determines if the probe is in active use (e.g., operating state of the ultrasound system is active use). Active probe use may be one of the possible operating states determined automatically at 302. If method 300 determines that the probe is in active use (YES) method 300 proceeds to 306 and includes continuing the ultrasound scan. In active use, it may be expected that the ultrasound probe face is in contact with gel acting as an interface between the ultrasound probe and the subject. For this reason, gel detected on a face of the ultrasound probe may be determined to be intentional and automatically detecting the presence of gel is not demanded. Method 300 returns.

If method 300 determines that the probe is not in active use (NO at 304), method 300 proceeds to 307 and includes automatically acquiring an image and/or cine sequence of the probe in air (e.g., the probe is not in active use so it is in air and not acoustically coupled to the patient). The probe may not be in active use when the probe is not actively being used to perform an ultrasound exam. Automatically acquiring the image may occur as a background process while the user is continuing to operate the ultrasound system with the probe either clean or dirty. The probe may not be in active use when the ultrasound system is in a pre-exam state and the user is preparing for the exam and performing such tasks as entering patient information. Further the probe may not be in active use when the ultrasound system is in an exam finished state and the user is finished with the exam and in the process of requesting a report.

Automatically acquiring the image and/or cine sequence may be triggered by the processor determining that the probe is no longer in active use and with no additional input from the user. The automatically acquired image may be stored in temporary memory of the ultrasound system and may be used for determining if the probe face is clean. The automatically acquired image may be stored for analysis as described below but may not be displayed to the user. In this way, limited display area is not taken up by images which are not of the patient and do not have clinical significance. In some examples, the display area may be limited when the display of the ultrasound system is a mobile device such as a tablet or laptop.

At 308, method 300 automatically determines if the probe face is clean. Automatically determining if the probe face is clean may include automatically analyzing the image and/or cine sequence that is acquired at step 307. Step 308 may also occur in the background while the user is operating the ultrasound system while the probe is either clean or dirty. In this way, the probe face is automatically checked for the presence of residual gel and/or water. A probe face of the ultrasound probe may be clean if it is dry and free of residual gel. The method may automatically determine if the probe face is clean based on automatic image analysis. The automatically analyzed images maybe an acquired near field image or cine sequence. The automatically analyzed images acquired from the ultrasound probe may be of the probe in air, before or after the probe is contacted with the imaging subject. In some embodiments, the ultrasound system 100 may or may not include additional physical sensors which may be used to automatically determine if the probe face is clean. In some embodiments, automatically determining if the probe face is clean may not include input from additional physical sensors coupled to the ultrasound probe face. For example, physical sensors may include moisture sensors or optical sensors. Use of additional sensors may demand additional power and processing allocation which may impair operating efficiency of the ultrasound system and reduce battery life in examples where the ultrasound system is a handheld ultrasound system.

Image analysis to determine if the probe is clean may occur based on signals sent to the processor by the transducers of the ultrasound probe and the images may not be displayed to the user. In some examples, the image processing algorithms may be machine learning algorithms. The image analyzed to determine if the probe is clean may be a near field image acquired by the ultrasound probe in air (e.g., not acoustically coupled to the patient).

Additionally or alternatively, a cine sequence captured by the ultrasound probe may be analyzed. A slow motion detected in the cine sequence may indicate a presence of residual gel or water on a face of the ultrasound probe. The speed of the motion may be distinguished from motion in the cine sequence indicative of a scan in progress. For example, a velocity of the image movement may be determined and velocity below a threshold velocity may indicate a presence of gel on the probe face. As another example, other algorithms such as correlation analysis and optical flow algorithms may be used to differentiate a cine sequence acquired from an ultrasound system in active use from a cine sequence acquired from a unclean probe face when the ultrasound system is not in active use. Image analysis algorithms are described further below with respect to FIG. 11 and a machine learning algorithm is further described in FIG. 12.

Turning briefly to FIGS. 6-9, examples of near field images acquired by an ultrasound probe in clean and dirty states are shown. FIG. 6 shows an example of a near field image 600 acquired by a clean dry ultrasound probe in air is shown. An ultrasound image acquired in air may refer to an ultrasound image acquired when a probe is spaced away from and not acoustically coupled to an imaging object. Near field image 600 includes a series of entrance echoes 602 caused by multiplier reflections at an acoustic stack of the probe. The entrance echoes may be present in a top portion of the acquired image and may decrease in brightness/contrast towards a vertical center of the image. A user may capture an image, such as near field image 600, for display on a monitor of the ultrasound system. In exemplary embodiment, pixel intensities associated with image 600 in addition to other near field images acquired when the ultrasound system is not in active use may be stored in a memory of the ultrasound system and analyzed to determine characteristics of the image formed by the pixels, but the near field image may not automatically be shown at a display of the ultrasound system. For example, when the ultrasound system is in a pre-exam state and not in an active use state, the display of the ultrasound may show a menu for entering patient information and may not show an ultrasound image, during this time the ultrasound system may acquire a near field image in the background to determine whether or not to display a warning to clean the probe.

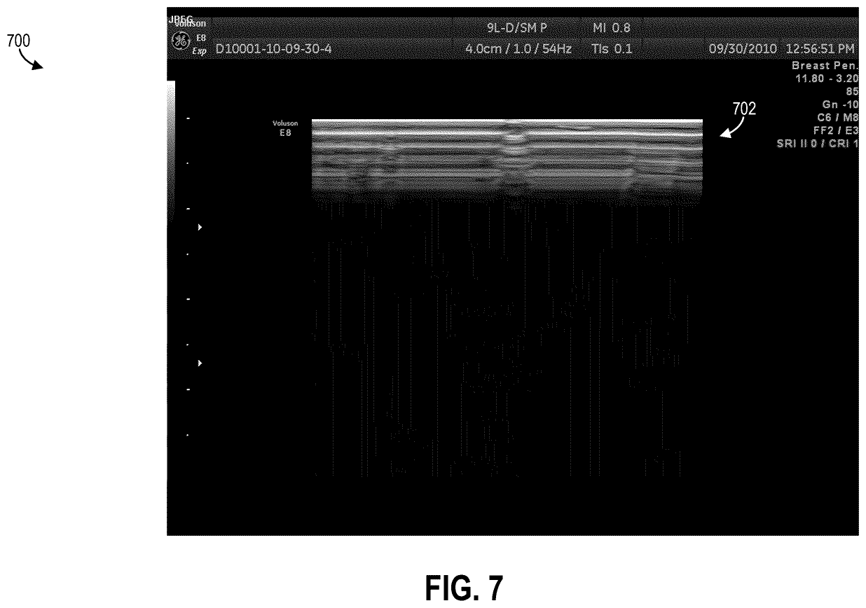

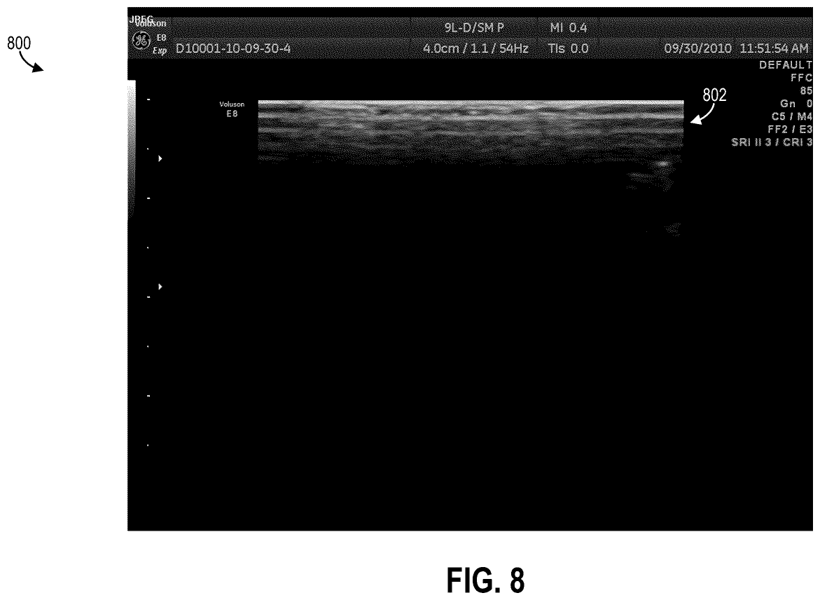

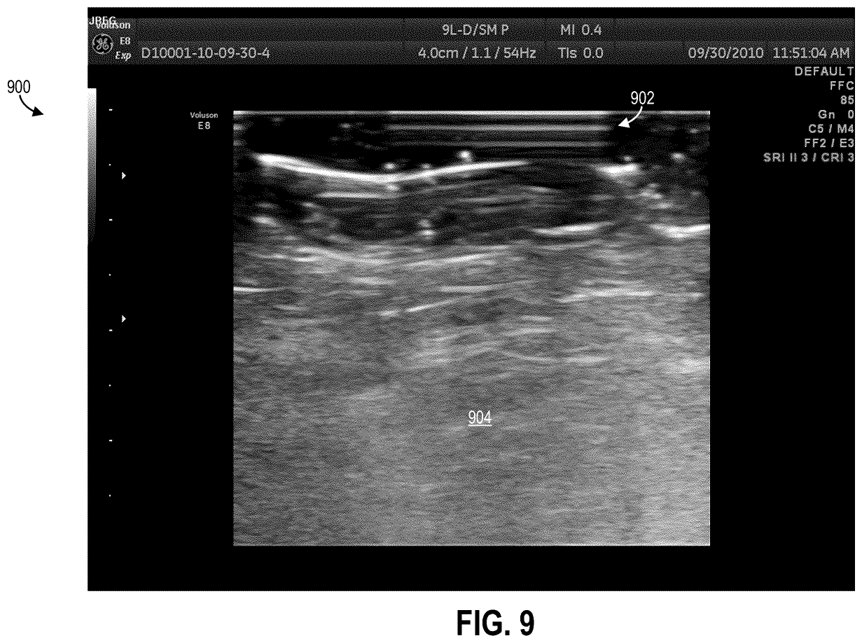

FIG. 7 shows a near field image 700 acquired by an ultrasound probe including residual water on a probe face. The edges of entrance echoes 702 of near field image 700 are disrupted due the presence of water. FIG. 8 shows a near field image 800 acquired by an ultrasound probe including a small amount of residual gel on the probe face. The edges of entrance echoes 802 of near field image 800 are blurred and not discrete as compared to entrance echoes 602 of the clean dry ultrasound probe. FIG. 9 shows a near field image 900 acquired by an ultrasound probe including a large amount of residual gel on the probe surface. Near field image 900 also includes entrance echoes 902 and also includes a large contrast area 904 which is not present in near field images 600, 700 or 800. Comparing near field images 600, 700, 800, and 900 may allow an image analysis algorithm to differentiate between a near field image acquired by a clean probe and a near field image acquired by a dirty probe.

In some examples, gel and/or water may be colorless and may not be apparent to the user by simple visual inspection. Image analysis to determine a presence of residual gel or water may determine the presence of gel that would otherwise not be noticed by the user. Additionally, in the examples shown in FIGS. 6-9, the differences between the reference image (FIG. 6) and the images acquired from the dirty ultrasound probe (FIGS. 7-9) may be noticed by a human observer. However, if the amount of residual gel is small, the difference between the reference image and the acquired image of the dirty ultrasound probe may be subtle and not readily noticed by observation alone. For example, only a small corner of the bars may be disrupted, or the movement in the cine sequence may be slow enough as to not be readily noticed by a human observer.

As one example, an image acquired from each probe of an ultrasound system may be acquired when the probe is clean and dry and used as a reference image. Image analysis may be used to compare the reference image to an acquired image to determine if the acquired image is of a clean probe face or a probe face with water and/or gel present. For example, total intensity of the image may be compared. As a further example, intensity over a depth of the image may be compared. As another example, near field images acquired by probes in clean and dirty states may be used to train a machine learning algorithm to differentiate between the two. As a further example, image analysis may use image filtering to look for blurring in the entrance echo bars present in the near field.

Turning now to FIG. 11, a method 1100 for automatically determining if an ultrasound probe face is clean (step 308 of method 300) is shown. Method 1100 may be at least partially performed on automatically acquired near field images after step 307 of FIG. 3.

At 1102, method 1100 includes comparing the acquired near field image (e.g., acquired at step 307) to a reference near field image. The reference near field image may be collected for each specific ultrasound probe used with an ultrasound system. The reference near field image may be collected probe is known to be clean and free of any residual water or gel. The ultrasound system may determine an identity of the ultrasound probe currently communicatively coupled to the ultrasound system and look up in non-volatile memory the corresponding reference near field image for the coupled probe.

In some examples, comparing the acquired near field image to the reference image may include comparing a total pixel intensity of the image. In alternate examples, comparing may include comparing pixel intensity of a identified rows and/or columns (e.g., line scan) of the near field images. In such examples intensity as a function of depth may be compared. As a further example, a difference in intensity of each pixel may be compared and a standard deviation of pixel intensity differences may be calculated.

At 1104, method 1100 determines if the acquired near field mage differs from the reference image by more than a threshold amount. The threshold amount may be set to a level low enough to correspond to the presence of residual gel on the probe surface. The threshold may be low enough to correspond to the presence of a small amount of gel on the probe surface which may otherwise not be visually noticed by the user. Further, the threshold amount may be set to a level high enough to prevent false positive identification of residual gel on the probe face.

If method 1100 determines that the images do not differ by more than the threshold amount (NO) (e.g., the probe face is clean), than method 1100 proceeds to 1106 and includes not performing a processor operation to indicate the probe face is not clean. If method 1100 determines that the images do differ by more than the threshold amount (YES), method 1100 proceeds to 1108 and includes performing the processor operation to indicate the probe face is not clean. The processor operation is discussed further below with respect to step 312 of method 300.

In addition to or alternatively to image analysis, artificial intelligence and machine learning may be used to automatically determine if the probe face is clean based on automatically acquired near field images or cine/sequences. A block diagram 1200 of an example algorithm for using machine learning to perform step 308 of method 300 is shown.

Training inputs 1202 may include a reference near field images and/or cine sequences 1204 collected in air using a plurality of clean ultrasound probes. The clean ultrasound probes may be free of residual gel and water. The same ultrasound probes used to collect the reference may then be purposefully dirtied and ground truth near field images and/or cine sequences of a dirty probe in air 1206 may be collected. Ultrasound probes may be made dirty with water and with residual gel. Additionally, varied amounts of residual ultrasound gel may be used. In some examples, the training inputs 1202 may include the entire ultrasound image and/or cine sequence collected in air and training inputs may not demand any further sectioning or identification from a skilled clinician. In this way, training the machine learning algorithm may be efficient in terms of man hours demanded for training.

Training inputs 1202 may be used to train identification algorithm 1210. Identification algorithm 1210 may be a machine learning algorithm such as linear regression, neural networks, or decision trees, among others. Acquired near field images and/or cine sequences 1208, such as those acquired at step 307 of method 300 may be fed into identification algorithm 1210. Identification algorithm 1210 may output an identification 1212 for the input near field image/cine sequence as being dirty of clean.

Returning now to FIG. 3, if method 300 determines that the probe is clean (YES), method 300 proceeds to 310 and includes proceeding to scanning or storing the probe. Proceeding to scanning may be done by the user if the ultrasound system was in a pre-exam state. When proceeding to scanning, the user may add fresh ultrasound gel to the patient and/or to the probe and the ultrasound system transitions to being an active scan state. In the active scan state, the method does not determine if the probe is clean. In some examples, the ultrasound system may be determined to be in idle state in between a pre-exam state and active scan state. As described further below with respect to FIG. 5, the method may allow a threshold elapsed time in the idle state before automatically determining if the probe face is clean. In this way, the user may transition between the pre-exam state and active scanning state without receiving a false warning that the probe face is not clean. Proceeding to storing the probe may be done by the user if all desired images of the subject are acquired and the ultrasound exam is ending. Method 300 returns.

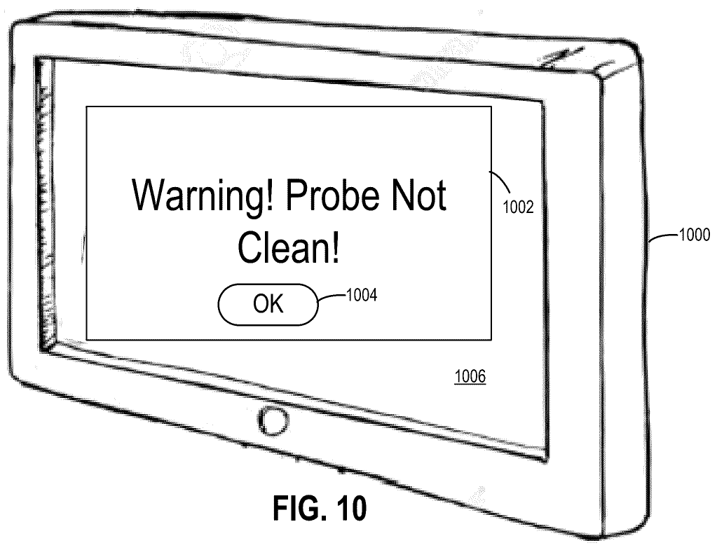

If method 300 determines that the probe is not clean (NO), method 300 proceeds to 312 and perform a processor operation to indicate that the probe is not clean and cleaning is demanded. As one example the processor operation may include displaying a warning on a display monitor of the ultrasound imaging system. An example of the warning is described further below with reference to FIG. 10. Additionally or alternatively, the warning may include flashing lights, loud sounds, a vibration, and/or other similar notifications. In further examples, the processor operation may include marking images acquired when the method determines that the probe face is not clean. For example, a label may be saved with an acquired ultrasound image indicating that the probe face was not clean. As one example, the label may be superimposed on the image reading “not clean”. The label may indicate to anyone viewing the acquired image that the user did not clean the ultrasound probe before acquiring the image.

In some examples the processor operation may prevent the user from acquiring an ultrasound image. For example, the processor may replace the displayed ultrasound image with video instructions on how to clean the probe. As a further example, the processor may set the probe in freeze mode. In the freeze mode, the transducer elements of the ultrasound probe may not be activated and therefore an image may not be acquired. In further examples, the processor may de-select the currently selected dirty probe so that it is no longer active in the ultrasound system. An ultrasound image may not be acquired without an active ultrasound probe. In some examples the processor may also automatically select a different probe. Additionally, after selecting a different probe, the processor may automatically determine if the new selected probe is clean as described above and proceed to select different ultrasound probes until a probe that is automatically determined to be clean is selected. In alternate examples, the processor may activate a vibration motor (e.g., vibration motor 216) of the probe handle until the probe is cleaned. The vibrations may both physically remind the user to clean the ultrasound probe and a constantly vibrating probe may be unable to acquire a readable ultrasound image.

In alternate examples, the processor may adjust settings of the ultrasound system which result in an unreadable ultrasound image. An unreadable ultrasound image may be poor contrast, blurry, fuzzy, or otherwise distorted so that a clinician may not be able to interpret the image. For example, the processor may turn down image brightness to the point where a contrast of the image is unreadable. As a further example, the processor may adjust beamforming delays to cause the image to be blurry to the point that the image is unreadable. As a further example, the processor may apply a low pass filter to the ultrasound image to cause the image to be unreadable.

At 314, method 300 includes waiting for a cleaning delay. The cleaning delay may be a duration of time during which method 300 gives the user time to clean the ultrasound probe. After waiting the cleaning delay, method 300 returns to 307 and again automatically acquires an image and/or cine sequence of the probe face in air. In this way, the method may check whether the user successfully cleaned the probe face. The user may continue to operate the ultrasound system with a dirty probe face while the delay occurs but may be stopped by one or more of the processor operations describe above with respect to step 312 once the method returns to step 307. The automatic image analysis as described above may be able to tell if the probe face is free of residual gel by identifying presence of residual gel that is missed, even when the user remembers or heeds the reminder to clean the ultrasound probe. Method 300 returns.

Turning now to FIG. 10, a display device of an ultrasound system 1000 is shown including a warning 1002. In some examples, warning 1002 may further include video instructions showing the user how to clean the probe face as described above. The display device may be equivalent to display device 118 of FIG. 1. In some examples, the display device may include two display devices, an image display device and a touch screen display device. The image display device may display the ultrasound image and the touch screen display may receive inputs from the user to operate the ultrasound system. As one example, warning 1002 may take up a majority (e.g., >50%) of a display area of the image display device or the touch screen display device. In one example, warning 1002 may take up a majority of the image display device and prevent a user from viewing an acquired ultrasound image. As a further example, warning 1002 may take up a majority of the touch screen display and may prevent the user from interacting with the user interface until the probe face is determined to be clean. Display area 1006 of the display device not including warning 1002 may show what was being viewed by the user when the warning was generated. The ultrasound system may analyze images acquired from the ultrasound probe to determine if the probe is clean without showing the acquired images to the user. In this way, an ultrasound image may not be displayed on the display area when the warning 1002 is present. For example, the user may be entering patient information or requesting a report and the menus related to the user actions may be obscured. In some examples, the warning 1002 may include an acknowledgement feature 1004. In such examples, clicking or otherwise acknowledging acknowledgement feature 1004 may clear warning 1002 and method 300 may proceed to wait the cleaning delay and then re-acquire the image or cine/sequence in air to determine if the probe face is dirty as described above.

Turning now to FIG. 5, a flowchart illustrating a method for determining when to check for a clean probe and when to remind a user to clean the probe is shown. The method may take into account that during an ultrasound exam the ultrasound system may be in an exam start, active use, idle, or exam finished state. In the exam start, idle, or exam finished states, the ultrasound system may automatically check if a probe face is clean. A detailed flowchart of a method 500 for determining when to display a warning to clean the probe depending on the determined state of the ultrasound system is shown. Herein, it is understood that to display a warning is an example of an operation performed by the processor to indicate the probe face is not clean. Other examples of operations are discussed above with respect to step 312 of method 300 and may be performed in addition to or instead of displaying a warning as described in method 500 below.

At 502, method 500 determines if the exam is started. As one example, a start of an exam may be determined by a user entering patient information into the user interface. As a further example, a start of an exam may be determined by an ultrasound probe being communicatively coupled to the ultrasound system and/or a sensor of the holder indicating movement of a probe out of the holder. If at 502, method 500 determines that the exam has not yet started (NO), method 500 proceeds to 504 and includes automatically determining if the probe is clean. In this way, the method determines if there is any residual gel left behind from a previous exam before the current exam starts. Determining if the probe is clean may include automatically acquiring an image and/or cine sequence of the probe in air and automatically analyzing the image as describe above with respect to FIG. 1, even if the image is not shown on the display of the ultrasound system. In this way the method may automatically check if the probe is clean while the user is performing other pre-exam tasks using the user interface and the limited display area may not be cluttered by additional images of the probe in air. If the probe is clean (YES), method 500 returns to 502. If the probe is not clean (NO), method 500 proceeds to 506 and includes warning the user that the probe is not clean. Method 500 returns. In some examples, the warning may be displayed and acquisition of images may be blocked until the system determines that the probe is clean. Other examples of processes performed by the processor in response to indicate the probe is not clean are discussed above with respect to step 312 of FIG. 3.

Returning to 502, if method 500 determines that the exam is started (YES), method 500 proceeds to 508 and includes determining if the probe is in active use. The probe may be in active use if the ultrasound system is determined to be in the active use state as described above. Determining if the probe is in active use may include receiving signals from the probe indicating that the probe is in motion. Additionally or alternatively, determining if the probe is in active use may include monitoring the images acquired as resulting from an imaging session in progress. If method 500 determines that the probe is in use (YES), method 500 proceeds to 510 and includes setting a first timer and a second timer to zero. As one example, the first timer may be used to monitor a duration that the ultrasound probe is idle and not in the holder and the second timer may be used monitor a time that the ultrasound probe is idle and placed within the holder. Method 500 returns to 508 and again determines if the probe is in use. In this way, the probe is not checked for cleanliness if the probe is in active use and the imaging session may not be unduly interrupted.

If at 508, method 500 determines that the probe is not in use (NO), method 500 proceeds to 512 and includes determining if the probe is in the holder. As one example, determining if the probe is in the holder may be based on a signal from a sensor of the ultrasound probe and/or the probe holder. If at 512, method 500 determines that the probe is in the holder (YES), method 500 proceeds to 514 and includes determining if the second timer is exceeded. The second timer may be exceeded if a count on the timer is higher than a threshold time. The threshold time may be a time that a user may be expected to rest an ultrasound probe in a holder during an imaging session. If at 514, method 500 determines that the second timer is not exceeded (NO), method 500 proceeds to 516 and includes adding a time increment to the second timer. Adding the time increment to the second timer may increase a time recorded on the second timer, moving the time closer to the threshold of the second timer. The time added may be equivalent to an elapsed time since the second timer was set to zero at 510. Method 500 then returns to 512 and again determines if the probe is in the holder.

If at 514, method 500 determines that the second timer is exceeded (YES), method 500 proceeds to 518 and determines if the probe is clean. Determining if the probe is clean at 518 may be similar to determining if the probe is clean at 504 and may include automatically analyzing images acquired from the ultrasound probe as described above with respect to step 308 of FIG. 3. If method 500 determines that the probe is not clean (NO), method 500 proceeds to 506 and warns/reminds the user to clean the probe. In this way, when the probe is positioned in the holder for a time exceeding the threshold time the method may assume the probe is no longer in use and demands cleaning. Method 500 returns.

If at 518, method 500 determines that the probe is clean (YES), method 500 proceeds to 520 and determines if the exam is ending. The exam ending may correspond to the ultrasound system being in the exam finished state as described above with respect to FIGS. 3-4. As one example, an exam ending may be determined by a user requesting a report of the exam. If at 520, method 500 determines that the exam is not ending (NO), method 500 returns to 510 and resets the first timer and second timer to zero. If the exam is ended (YES), method 500 proceeds to 522 and finishes the exam, method 500 returns. If at 520, method 500 determines that the exam is ending (YES), method 500 proceeds to 522 and includes finishing the exam. Method 500 returns.

Returning to 512, if method 500 determines that the probe is not in the holder (NO), method 500 proceeds to 524 and includes determining if the first timer is exceeded. Exceeding the first time may include the first timer exceeding a threshold time of the first timer. The threshold time of the first timer may be an expected time for the ultrasound system to be in the idle state as determined by method 400 while the ultrasound probe is not in the holder. For example, the user may not be actively using the probe but may be preparing to measure a different portion of the subject. If the first timer is not exceeded (NO), method 500 proceeds to 526 and includes incrementing the first timer. Incrementing the first timer may include adding a time to the first timer equivalent to an elapsed time since the first timer was set to zero at step 510. Method 500 then continue to 508 and again determines if the probe is in use. If at 524, method 500 determines that the first timer is exceeded (YES), method 500 proceeds to 518 and again determines if the probe is clean and proceeds as described above. In this way, when the ultrasound system is in an idle state and the ultrasound probe is not in the holder for a duration exceeding the first timer threshold, the system may automatically determine that the ultrasound system is entering an exam finished state and may check to see if the probe is cleaned.

The technical effect of methods 300, 400, 500, and 1100 are to automatically and dynamically determine when to display a warning/reminder to a user of an ultrasound system to clean a probe face. In this way, the user is warned in situations where residual gel and/or water is detected and not expected to be present. Warning the user in states where the warning is demanded and not in states where the warning is not demanded (e.g., the probe is already clean or actively in use) may increase compliance with the warning and prevent the warning becoming like background noise. Even if the user does remember to clean the probe, the user may not clean the probe thoroughly and leave behind residual gel that is not noticeable or visible to the user. Automatically determining if the probe is clean using automatic image analysis detects residual gel that may not be otherwise visible to the human eye and may do so as a background process that does not take up limited display space. Further, the automatic image analysis may detect the small differences in the images caused by residual gel that may not be noticed by a human user. Additionally, the system described herein may use components included in an ultrasound system and may not demand adoption of new hardware or additional sensors to implement the methods. Further, image analysis offers advantages in terms of saved processing power over addition of physical sensors, such as moisture or optical sensors, to detect residual gel. Additional sensors may be slow to turn off/on and may demand additional processing power and battery power (in the case of a wireless ultrasound probe) to continuously run in the background. Increasing user compliance with cleaning a probe face of residual gel may help prevent transmission of infection vectors via ultrasound gel.

The disclosure also provides support for an ultrasound system, comprising: an ultrasound probe comprised of transducer elements comprised of piezoelectric material, a damping block positioned behind the transducer elements, a matching layer positioned in front of the transducer elements, and a sensor configured to detect motion of the ultrasound probe, a holder comprising a sensor configured to detect motion of the ultrasound probe into and/or out of the holder, wherein the ultrasound probe is positioned inside the holder when the ultrasound system is not in an active use state, and a processor communicatively coupled to a display and a user interface, wherein the processor includes instructions stored on non-volatile memory that when executed cause the processor to: determine an operating state of the ultrasound system, in response to determining the operating state is not the active use state, automatically determine by image analysis if a probe face of the ultrasound probe is clean, and in response to determining the probe face is not clean, perform a processor operation to prevent acquiring an image with the ultrasound probe. In a first example of the system, the instructions further cause the processor to determine user interaction with the user interface, monitor the sensor of the ultrasound probe and/or the holder, and analyze images received from the ultrasound probe to determine the operating state of the ultrasound system. In a second example of the system, optionally including the first example, the operating state of the ultrasound system is one of active use, idle, pre-exam, and exam finished. In a third example of the system, optionally including one or both of the first and second examples, a clean probe face is free of water and residual gel. In a fourth example of the system, optionally including one or more or each of the first through third examples, to automatically determine by image analysis if the probe face is clean, an image or cine sequence is acquired of the probe face in air. In a fifth example of the system, optionally including one or more or each of the first through fourth examples, the acquired image or cine sequence is not displayed to on the display of the ultrasound system. In a sixth example of the system, optionally including one or more or each of the first through fifth examples, the processor operation includes one or more of adjusting settings of the ultrasound system to cause an unreadable ultrasound image, adjusting the ultrasound probe to a freeze mode, de-selecting the ultrasound probe, and activating a vibration motor positioned within a handle of the ultrasound probe. In a seventh example of the system, optionally including one or more or each of the first through sixth examples, the instructions further include to not perform the processor operation in response to determining the probe face is clean.

The disclosure also provides support for an ultrasound system, comprising: an ultrasound probe, a holder, wherein the ultrasound probe is positioned inside the holder when the ultrasound system is not in an active use state, and a processor communicatively coupled to a display and a user interface, wherein the processor includes instructions stored on non-volatile memory that when executed cause the processor to: automatically identify an operating state of the ultrasound system, wherein the operating state is one of active use, idle, pre-exam, or exam finished, in response to the ultrasound system in the pre-exam state or exam finished state automatically determining if a probe face of an ultrasound probe of the ultrasound system is clean, in response to the ultrasound system in the idle state, automatically determining if the probe face is clean if a timer is exceeded, in response the ultrasound system in the active use state, setting the timer to zero and not automatically determining if the probe face is clean, if the probe face is determined to not be clean, perform a processor operation to indicate that the probe face of the ultrasound probe is not clean, wait for a cleaning delay and then automatically determine if the probe face is clean, and when the probe face is determined to be clean, not performing the processor operation. In a first example of the system, automatically determining if the probe face is clean includes acquiring a near field image and/or cine sequence using the ultrasound probe in air and automatically analyzing the near field image and/or cine sequence. In a second example of the system, optionally including the first example, automatically analyzing the near field image includes comparing a top portion of the near field image including bars caused by entrance echoes. In a third example of the system, optionally including one or both of the first and second examples, automatically analyzing the cine sequence includes determining a rate of motion in the cine sequence is present and the rate of motion is slower than a rate of motion of the cine sequence when the ultrasound system is in the active use state. In a fourth example of the system, optionally including one or more or each of the first through third examples, the system further comprises: not displaying the acquired near field image and/or cine sequence on the display of the ultrasound system. In a fifth example of the system, optionally including one or more or each of the first through fourth examples, the processor operation includes one or more of generating a warning at the display of the ultrasound system, replacing an ultrasound image with video instructions for cleaning the ultrasound probe, and saving an ultrasound image with a label indicating the ultrasound probe was not clean when the ultrasound image was acquired.

The disclosure also provides support for a method for an ultrasound system, comprising: continuing the ultrasound system in an inactive state, while automatically acquiring an image and/or cine sequence of a probe of the ultrasound system in air, automatically identify, using image analysis of the automatically acquired image and/or cine sequence, that a face of the probe is not clean and, performing an operation using a processor of the ultrasound system to indicate that the face of the probe is not clean. In a first example of the method, the method further comprises: automatically identifying an operating state of the ultrasound system by one or more of determining user interaction with a user interface, monitoring sensors of the ultrasound system, or analyzing received images. In a second example of the method, optionally including the first example, automatically identifying the operating state of the ultrasound system includes monitoring a motion sensor of the probe and/or a sensor of a holder of the ultrasound system. In a third example of the method, optionally including one or both of the first and second examples, automatically identifying the operating state of the ultrasound system includes analyzing received images including monitoring a cine sequence and/or monitoring an intensity of far field images. In a fourth example of the method, optionally including one or more or each of the first through third examples, the method further comprises: when the operating state of the ultrasound system is in active use, continuing scanning and not determining if the face of the probe is clean. In a fifth example of the method, optionally including one or more or each of the first through fourth examples, automatically determining if the face of the probe is clean includes analyzing entrance echoes of the image of the probe in air.

FIG. 2 shows an example configuration with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An ultrasound system, comprising:
an ultrasound probe comprised of transducer elements comprised of piezoelectric material, a damping block positioned behind the transducer elements, a matching layer positioned in front of the transducer elements, and a sensor configured to detect motion of the ultrasound probe;
a holder comprising a sensor configured to detect motion of the ultrasound probe into and/or out of the holder, wherein the ultrasound probe is positioned inside the holder when the ultrasound system is not in an active use state; and
a processor communicatively coupled to a display and a user interface, wherein the processor includes instructions stored on non-volatile memory that when executed cause the processor to:
determine an operating state of the ultrasound system;
in response to determining the operating state is not the active use state, automatically determine by image analysis if a probe face of the ultrasound probe is clean; and
in response to determining the probe face is not clean, perform a processor operation to indicate the probe face is not clean.

2. The ultrasound system of claim 1, wherein the instructions further cause the processor to determine user interaction with the user interface, monitor the sensor of the ultrasound probe and/or the holder, and analyze images received from the ultrasound probe to determine the operating state of the ultrasound system.

3. The ultrasound system of claim 1, wherein the operating state of the ultrasound system is one of active use, idle, pre-exam, and exam finished.

4. The ultrasound system of claim 1, wherein a clean probe face is free of water and residual gel.

5. The ultrasound system of claim 1, wherein to automatically determine by image analysis if the probe face is clean, an image or cine sequence is acquired of the probe face in air.

6. The ultrasound system of claim 5, wherein the acquired image or cine sequence is not displayed on the display of the ultrasound system.

7. The ultrasound system of claim 1, wherein the processor operation includes one or more of adjusting settings of the ultrasound system to cause an unreadable ultrasound image, adjusting the ultrasound probe to a freeze mode, de-selecting the ultrasound probe, and activating a vibration motor positioned within a handle of the ultrasound probe.

8. The ultrasound system of claim 1, wherein the instructions further include to not perform the processor operation in response to determining the probe face is clean.

9. An ultrasound system, comprising:
an ultrasound probe;
a holder, wherein the ultrasound probe is positioned inside the holder when the ultrasound system is not in an active use state; and
a processor communicatively coupled to a display and a user interface, wherein the processor includes instructions stored on non-volatile memory that when executed cause the processor to:
automatically identify an operating state of the ultrasound system, wherein the operating state is one of active use, idle, pre-exam, or exam finished;
in response to the ultrasound system in the pre-exam state or exam finished state automatically determining if a probe face of an ultrasound probe of the ultrasound system is clean;
in response to the ultrasound system in the idle state, automatically determining if the probe face is clean if a timer is exceeded;
in response the ultrasound system in the active use state, setting the timer to zero and not automatically determining if the probe face is clean;
if the probe face is determined to not be clean, perform a processor operation to indicate that the probe face of the ultrasound probe is not clean, wait for a cleaning delay and then automatically determine if the probe face is clean; and
when the probe face is determined to be clean, not performing the processor operation.

10. The ultrasound system of claim 9, wherein automatically determining if the probe face is clean includes acquiring a near field image and/or cine sequence using the ultrasound probe in air and automatically analyzing the near field image and/or cine sequence.

11. The ultrasound system of claim 10, wherein automatically analyzing the near field image includes comparing a top portion of the near field image including bars caused by entrance echoes.

12. The ultrasound system of claim 10, wherein automatically analyzing the cine sequence includes determining a rate of motion in the cine sequence is present and the rate of motion is slower than a rate of motion of the cine sequence when the ultrasound system is in the active use state.

13. The ultrasound system of claim 10, further comprising not displaying the acquired near field image and/or cine sequence on the display of the ultrasound system.

14. The ultrasound system of claim 10, wherein the processor operation includes one or more of generating a warning at the display of the ultrasound system, replacing an ultrasound image with video instructions for cleaning the ultrasound probe, and saving an ultrasound image with a label indicating the ultrasound probe was not clean when the ultrasound image was acquired.

15. A method for an ultrasound system, comprising:
continuing the ultrasound system in an inactive state, while automatically acquiring an image and/or cine sequence of a probe of the ultrasound system in air, automatically identify, using image analysis of the automatically acquired image and/or cine sequence, that a face of the probe is not clean and, performing an operation using a processor of the ultrasound system to indicate that the face of the probe is not clean.

16. The method of claim 15, further comprising automatically identifying an operating state of the ultrasound system by one or more of determining user interaction with a user interface, monitoring sensors of the ultrasound system, or analyzing received images.

17. The method of claim 16, wherein automatically identifying the operating state of the ultrasound system includes monitoring a motion sensor of the probe and/or a sensor of a holder of the ultrasound system.

18. The method of claim 16, wherein automatically identifying the operating state of the ultrasound system includes analyzing received images including monitoring a cine sequence and/or monitoring an intensity of far field images.

19. The method of claim 16, further comprising when the operating state of the ultrasound system is in active use, continuing scanning and not determining if the face of the probe is clean.

20. The method of claim 15, wherein automatically determining if the face of the probe is clean includes analyzing entrance echoes of the image of the probe in air.
